(19) United States
(12) Patent Application Publication
(10) Pub. No.: US 2023/0181920 A1
(43) Pub. Date: Jun. 15, 2023

(54) TREATMENT OF LYME DISEASE USING HIGH-INTENSITY FOCUSED ELECTROMAGNETIC ENERGY

(71) Applicant: Gregory Derderian, Grand Blanc, MI (US)

(72) Inventor: Gregory Derderian, Grand Blanc, MI (US)

(21) Appl. No.: 18/077,774

(22) Filed: Dec. 8, 2022

Related U.S. Application Data
(60) Provisional application No. 63/289,397, filed on Dec. 14, 2021.

(51) Int. Cl. A61N 2/00; A61N 2/02

(52) U.S. Cl. CPC A61N 2/004; A61N 2/002; A61N 2/02

(57) ABSTRACT

A method of treating an illness caused by microbial infection includes administering a first high intensity focused electromagnetic field treatment to a patient whereby a first Herx reaction is induced in the patient; and administering a second high intensity focused electromagnetic field treatment to the patient after the first Herx reaction has subsided, whereby a second Herx reaction is induced in the patient and the second Herx reaction has a lower duration or intensity than the first Herx reaction. A method of treating an illness caused by microbial infection is also disclosed.

### CROSS-REFERENCE TO RELATED APPLICATIONS

This application claims priority to U.S. Provisional Application 63/289,397 filed Dec. 14, 2021, which is hereby incorporated by reference herein in its entirety.

### BACKGROUND

The present application relates to treatments for microbial diseases, such as but not limited to, bacterial diseases including Lyme disease. More specifically, the present invention relates to the use of high-intensity electromagnetic energy in the treatment of Lyme disease as well as other microbial diseases not limited to bartonella, babesia and other Gram positive and Gram negative microbes.

Lyme disease is the most common vector-borne disease in the United States. Lyme disease is caused by four main species of bacteria—Borrelia burgdorferi and Borrelia mayonii in the United States, and Borrelia afzelii and Borrelia garinii in Europe and Asia. It is typically transmitted to humans through the bite of an infected black—legged tick, commonly known as a deer tick.

The signs and symptoms of Lyme disease often vary from person to person. In the early stages—within 30 days of infection—a rash with an expanding red are surrounding a clear area in the middle may appear forming what some describe as a bull's eye pattern. Over time a plethora of other symptoms occur which include but are not limited to myalgia, joint pain, fever, headache, neurologic symptoms, fatigue, reproductive abnormalities, and a characteristic skin rash called erythema migrans. If left untreated, the infection can spread causing new and exacerbating old symptoms. In certain cases Lyme disease can be fatal, as in the case of Lyme carditis.

Many of the symptoms of Lyme disease are found in other conditions, making diagnosis difficult. Lyme disease is diagnosed based on symptoms, physical findings (e.g., rash), and the possibility of exposure to infected ticks. Laboratory testing can be helpful but is unreliable and fraught with false negatives.

Antibiotics are commonly used to treat Lyme disease. Generally, recovery will be quicker and more complete if treatment is begun early. A 14-21 day course or oral antibiotics is usually recommended for early Lyme disease infections. Chronic Lyme disease is much more difficult to treat. Treatment may include oral and or IV antibiotics as well as a variety of alternative therapies. Often treatment is considered successful if the patient's symptoms are ameliorated and they can live a productive life.

Borrelia Burgdorferi is a gram-positive spirochete that which if not completely eradicated can lead to Post-Treatment Lyme Disease Syndrome (PTLDS) or Chronic Lyme Disease (CLD). If patients fail treatment or are not recognized and treated initially, these patients can have a multitude of symptoms including, but not limited to, pain, fatigue, or difficulty thinking that lasts indefinitely.

According to the CDC, there is no proven treatment for PTLDS or CLD. Although short-term antibiotic treatment is a proven treatment for early Lyme disease, studies funded by the National Institutes of Health (NIH) suggest that long-term outcomes are no better for patients who received additional prolonged antibiotic treatment than for patients who received placebo. Long-term antibiotic treatment for Lyme disease allegedly has been associated with serious, sometimes deadly complications.

Accordingly, there is a need for a proven, effective treatment for Post-Treatment Lyme Disease Syndrome (PTLDS) and/or Chronic Lyme Disease (CLD) that is safe and effective.

### SUMMARY OF THE INVENTION

A method of treating an illness caused by microbial infection according to an exemplary embodiment of this disclosure, among other possible things includes administering a first high intensity focused electromagnetic field treatment to a patient whereby a first Herx reaction is induced in the patient; and administering a second high intensity focused electromagnetic field treatment to the patient after the first Herx reaction has subsided, whereby a second Herx reaction is induced in the patient and the second Herx reaction has a lower duration or intensity than the first Herx reaction.

In a further example of the foregoing, the method includes administering a third high intensity focused electromagnetic field treatment to the patient after the second Herx reaction has subsided, whereby a third Herx reaction is induced in the patient and the third Herx reaction has a lower duration or intensity than the second Herx reaction.

In a further example of any of the foregoing, the second treatment is administered after the Herx reaction has subsided between about 1 and about 50 days after the first treatment.

In a further example of any of the foregoing, the first and second treatments have a duration of about 30 minutes.

In a further example of any of the foregoing, the first and second treatments are administered to at least one of the patient's abdomen and buttocks.

In a further example of any of the foregoing, an intensity of each of the first and second treatments is between about 0.5 T and about 4 T.

In a further example of any of the foregoing, the intensity of each of the first and second treatments is between about 0.5 T and about 2 T.

In a further example of any of the foregoing, a frequency of an electromagnetic wave used during each of the first and second treatments is in the super low frequency (SLF) or extremely low frequency (ELF) range.

In a further example of any of the foregoing, a frequency of an electromagnetic wave used during each of the first and second treatments is between about 5 Hz and 100 Hz.

In a further example of any of the foregoing, the method includes administering a cycle of more than two high intensity focused electromagnetic field treatments to the patient.

In a further example of any of the foregoing, the cycle includes between about 10 and about 20 high intensity focused electromagnetic field treatments.

In a further example of any of the foregoing, each of the treatments in the cycle has an intensity between about 1.8 T and about 4 T.

In a further example of any of the foregoing, each of the treatments in the cycle has a duration of about 30 minutes.

In a further example of any of the foregoing, a frequency of an electromagnetic wave used during each of the first and second treatments is in the super low frequency (SLF) or extremely low frequency (ELF) range.

In a further example of any of the foregoing, each of the treatments are administered to each of the patient's abdomen, buttocks, thighs, arms, and calves.

In a further example of any of the foregoing, at least one of the treatments is administered with radiofrequency.

In a further example of any of the foregoing, at least one of the treatments is administered with radiofrequency.

In a further example of any of the foregoing, the method includes administering antibiotics to the patient.

A method of treating an illness caused by microbial infection according to an exemplary embodiment of this disclosure, among other possible things includes administering a cycle of high intensity focused electromagnetic field treatments to a patient, whereby the treatments decrease the population of at least one microbe in the patient.

In a further example of the foregoing, at least one of the treatments is administered with radiofrequency.

### DETAILED DESCRIPTION

High intensity focused electromagnetic field technology, in some cases with radiofrequency (RF) has been used in the field of aesthetic medicine for muscle toning and strengthening, body contouring, and fat reduction. In general, in this application, high intensity focused electromagnetic field treatment mimics the action potential of nerves that cause muscle contractions.

It is postulated that efficacious therapy for Post-Treatment Lyme Disease Syndrome (PTLDS) and/or Chronic Lyme Disease (CLD) results in death of the bacteria, and consequent release of bacterial toxins into the bloodstream, leading to a systemic inflammatory response. Depending on the patients bacterial load this response can develop into a Jarisch-Herxheimer (Herx) reaction which temporarily overwhelms the bodies' ability to clear the toxins. Herx reactions often manifest by magnifying a patient's PTLDS or CLD symptoms and can be significantly debilitating. A patient's Herx reaction is identified and quantified by self-reporting of the duration and severity of symptoms.

It has been discovered that a series of high intensity focused electromagnetic field treatments of a duration and intensity to initially induce a Herx reaction is efficacious in treating illnesses caused by microbial infection such as Lyme disease, including PTLDS or CLD. Specifically, for patients with PTLDS or CLD, it has been discovered that the Herx reactions in patients with PTLDS or CLD decrease in intensity and/or duration after each subsequent high intensity focused electromagnetic field treatment. The high intensity focused electromagnetic field treatment decrease microbe populations in the patient. Without being bound by any particular theory, it is thought that the number of colony-forming units of certain microbes decreases with time of exposure to rapidly changing magnetic fields such as those used in high intensity focused electromagnetic field treatments.

The high intensity focused electromagnetic field treatment includes a series or of treatments known as a cycle. Each treatment is performed with a high intensity focused electromagnetic field device, which are known in the art, for a predetermined duration and at predetermined intensity. One example high intensity focused electromagnetic field device is the EMSCULPT® or EMSCULPT NEO® device (BTL Industries, Inc., Marlborough, MA). The high intensity focused electromagnetic field treatment may be administered with or without radiofrequency. For instance, a cycle may consist of about 10 to about 20 treatments delivered about 1 to about 50 days apart, over several months. In a particular example, the treatments may be delivered about 3 to about 5 days apart.

Each treatment may be up to about 60 minutes long. In a particular, example, the treatments last for about 30 minutes.

In a particular example, the treatment may simultaneously occur in one, several, or all of the following areas of the patients' body: abdomen, buttocks, thighs, aims, and calves. In a particular example, the treatments are delivered to at least one of the abdomen and buttocks.

By the end of the treatment cycle, the Herx reactions are eliminated or greatly reduced and the PTLDS or CLD symptoms are significantly lessened or gone entirely.

In one example, the intensity of each treatment is between about 0.5 T and about 4 T. In a further example, the intensity of each treatment is between about 0.5 and about 1.8 T. IN another example, the intensity of each treatment is between about 1.8 T and about 4 T. In a further example, the intensity of each treatment is between about 1.5 T and about 2 T. The intensity of each treatment in a cycle can be the same or different.

In one example, the frequency of an electromagnetic wave used during each treatment is in the super low frequency (SLF) or extremely low frequency (ELF) range, and is between about 5 Hz and 100 Hz.

After administration of the first high intensity focused electromagnetic field treatment, the patient experiences a significant Herx reaction within hours of the treatment which may last for several (about 1 to about 14) days.

Once the Herx reaction has subsided, meaning the patient is not reporting Herx symptoms, for at least about 1 but up to about 3 days, a second high intensity focused electromagnetic field treatment can be administered. After the second treatment, the patient will again experience a Herx reaction, but that reaction will usually be less severe and/or will be shorter in duration than the Herx reaction that occurred after the first treatment. Additional high intensity focused electromagnetic field treatments can continue to be administered upon the dissipation of each successive Herx reaction until the patent no longer experiences a Herx reaction. At that point, additional high intensity focused electromagnetic field treatments can be administered until the patient's symptoms are significantly reduced or until they have remitted entirely.

### PATIENT EXAMPLE 1

One patient having a pre-treatment Horowitz Lyme-MSIDS questionnaire score of 137 points+25 points for the Incidence score, a series of twelve (12) high intensity focused electromagnetic field treatments treatments were administered over a period of time. The initial treatment included a full 30-minute treatment progressing to a 100% energy (1.8 Tesla) output. Shortly after treatment, the patient noted symptoms of a mild Herx reaction with chills. Several hours later the patient developed a severe Herx reaction with mood swings and visual disturbance accompanied by pronouncement of the patient's other symptoms. These symptoms persisted for 5 days. During this time the patient used oral supplements for liver detoxification and lymphatic support. Following the recovery, a second treatment was performed forty-two (42) days after the first treatment. Full therapeutic time and (30 minutes) energy levels (1.8 Tesla) were attained. Within hours the patient had a Herx reaction lasting 5 days but the intensity was much less pronounced. In total the subject underwent twelve (12) treatments all for 30 minutes at 100% energy (1.8 Tesla). After treatments 4-8 Herx reactions were less intense lasting for a day to a few hours and patient's overall feeling of well-being continued to improve. Following the 8th treatment sixty-eight (68) days after the first treatment, the patient had complete resolution of the neurologic (brain fog and mood swings) and musculoskeletal symptoms, peripheral neuropathy, and bone pain. However, the stiffness of the 3^{rd} finger of the left hand persisted. The patient's Horowitz Lyme score eighty-two (82) days after the initial treatment decreased to 1 point.

Subsequently, the patient had gallbladder surgery and did not have treatments for 3 weeks during which time patient had mild recurrence of symptoms but not nearly as severe as previously. With the last four treatments, the patient did not have Herx reactions, but did have continued improvement in overall feeling of wellbeing. The last treatment was performed one-hundred thirty-three (133) days after the first treatment at which time the patient had resolution of all issues with a Horowitz Lyme score of 0. Two hundred and seventy (270) days after the patient's last treatment, she remains asymptomatic. The patient is now able to clench fists, extend fingers and grip a zip line and all other symptoms have abated.

### PATIENT EXAMPLE 2

A second patient completed high intensity focused electromagnetic field treatments with no Herx reaction after the tenth treatment. The patient had subsequent eleventh high intensity focused electromagnetic field treatments with radiofrequency and after this treatment did have a Herx reaction and a subsequent improved sense of well-being. Microscopy was performed after certain of the treatments and a decrease in multiple microbes was noted after subsequent treatments.

The duration, intensity, and number of treatments, as well as the time between treatments can be varied to suit individual patient needs in accordance with the scope and spirit of the present invention.

In some examples, antibiotics may be administered during a treatment cycle. The antibiotics work in tandem with the treatments to combat bacterial disease. The antibiotics may be administered according to any known regimen depending on the character of the antibiotics.

As used herein, the term “about” has the typical meaning in the art, however in a particular example “about” can mean deviations of up to 10% of the values described herein.

Although a combination of features is shown in the illustrated examples, not all of them need to be combined to realize the benefits of various embodiments of this disclosure. In other words, a system designed according to an embodiment of this disclosure will not necessarily include all of the features shown in any one of the figures or all of the portions schematically shown in the figures. Moreover, selected features of one example embodiment may be combined with selected features of other example embodiments.

Although an embodiment of this invention has been disclosed, a worker of ordinary skill in this art would recognize that certain modifications would come within the scope of this disclosure. For that reason, the following claims should be studied to determine the true scope and content of this disclosure.

What is claimed is:

1. A method of treating an illness caused by microbial infection, comprising:
administering a first high intensity focused electromagnetic field treatment to a patient whereby a first Herx reaction is induced in the patient; and
administering a second high intensity focused electromagnetic field treatment to the patient after the first Herx reaction has subsided, whereby a second Herx reaction is induced in the patient and the second Herx reaction has a lower duration or intensity than the first Herx reaction.

2. The method of claim 1, further comprising administering a third high intensity focused electromagnetic field treatment to the patient after the second Herx reaction has subsided, whereby a third Herx reaction is induced in the patient and the third Herx reaction has a lower duration or intensity than the second Herx reaction.

3. The method of claim 1, wherein the second treatment is administered after the Herx reaction has subsided between about 1 and about 50 days after the first treatment.

4. The method of claim 1, wherein the first and second treatments have a duration of about 30 minutes.

5. The method of claim 1, wherein the first and second treatments are administered to at least one of the patient's abdomen and buttocks.

6. The method of claim 1, wherein an intensity of each of the first and second treatments is between about 0.5 T and about 4 T.

7. The method of claim 6, wherein the intensity of each of the first and second treatments is between about 0.5 T and about 2 T.

8. The method of claim 1, wherein a frequency of an electromagnetic wave used during each of the first and second treatments is in the super low frequency (SLF) or extremely low frequency (ELF) range.

9. The method of claim 1, wherein a frequency of an electromagnetic wave used during each of the first and second treatments is between about 5 Hz and 100 Hz.

10. The method of claim 1, further comprising administering a cycle of more than two high intensity focused electromagnetic field treatments to the patient.

11. The method of claim 10, wherein the cycle includes between about 10 and about 20 high intensity focused electromagnetic field treatments.

12. The method of claim 11, wherein each of the treatments in the cycle has an intensity between about 1.8 T and about 4 T.

13. The method of claim 12, wherein each of the treatments in the cycle has a duration of about 30 minutes.

14. The method of claim 13, wherein a frequency of an electromagnetic wave used during each of the first and second treatments is in the super low frequency (SLF) or extremely low frequency (ELF) range.

15. The method of claim 14, wherein each of the treatments are administered to each of the patient's abdomen, buttocks, thighs, arms, and calves.

16. The method of claim 15, wherein at least one of the treatments is administered with radiofrequency.

17. The method of claim 1, wherein at least one of the treatments is administered with radiofrequency.

18. The method of claim 1, further comprising administering antibiotics to the patient.

19. A method of treating an illness caused by microbial infection, comprising:
administering a cycle of high intensity focused electromagnetic field treatments to a patient, whereby the treatments decrease the population of at least one microbe in the patient.

20. The method of claim 19, wherein at least one of the treatments is administered with radiofrequency.
